(19) United States
(12) Patent Application Publication
(10) Pub. No.: US 2015/0361419 A1
(43) Pub. Date: Dec. 17, 2015

(54) MICROFLUIDIC CHIP FOR EXTRACTING NUCLEIC ACIDS, DEVICE FOR EXTRACTING NUCLEIC ACIDS COMPRISING SAME, AND METHOD FOR EXTRACTING NUCLEIC ACIDS USING SAME

(71) Applicant: NANOBIOSYS INC., Geumcheon-gu Seoul (KR)

(72) Inventors: Sung Woo KIM, Seoul (KR); Duck Joong KIM, Anyang-si, Gyeonggi-do (KR); Dong Hoon LEE, Anyang-si, Gyeonggi-do (KR); Sun Jin KIM, Seoul (KR); Yong Hea CHOI, Seoul (KR); Ho Sun RYU, Seoul (KR)

(21) Appl. No.: 14/762,182

(22) PCT Filed: Jan. 21, 2013
(86) PCT No.: PCT/KR2013/000481

§ 371 Date: Jul. 20, 2015

(51) Int. Cl. C12N 15/10; B01L 3/00; C12Q 1/68

(52) U.S. Cl. CPC C12N 15/1017; C12Q 1/6806; B01L 3/502715; B01L 3/502753; B01L 2300/0877; B01L 2300/12; B01L 2300/1805; B01L 2300/0681; B01L 2200/0631

(57) ABSTRACT

The present invention relates to a microfluidic chip for extracting nucleic acids, a nucleic acid extraction device having the same, and a nucleic acid extraction method using the same that can provide micro-miniaturization and ultra high speed, while maintaining and/or improving reliable nucleic acid extraction efficiencies, unlike the existing nucleic acid extraction device and method.

### TECHNICAL FIELD

The present invention relates to a microfluidic chip and a nucleic acid extraction device, and a method for extracting nucleic acids from a biological sample like cells, bacteria or viruses.

### BACKGROUND ART

For diagnosis, treatment, or prevention of diseases at the genetic level, techniques for extracting nucleic acids from a biological specimen such as cells, bacterium, or viruses have recently been in wide use in association with the nucleic acid amplification techniques. The techniques for extracting nucleic acids from the biological specimen are also on demand in various fields of applications, such as customized drug development, forensic, detection of endocrine disruptors, and so forth.

An example of the conventional nucleic acid extraction techniques is a method of solubilizing a specimen including cells with SDS or proteinase K, modifying and removing proteins with phenol, and then purifying a nucleic acid. However, the phenol-based extraction method has a credibility problem because the phenol-based extraction method requires a number of steps, which takes a lot of time, and the efficiency of the nucleic acid extraction method greatly depends on the worker's experience and skills. To resolve this problem, a kit having silica or glass fiber that specifically combines with a nucleic acid has been recently used. The silica or glass fiber has a low combining ratio with proteins or cell metabolites, so it is possible to acquire a nucleic acid at a relatively high concentration. This method is advantageous because it is more convenient in comparison to the phenol-based method. But the method uses a chaotropic reagent or ethanol that strongly inhibits the enzyme reaction such as polymerization chain reaction (PCR) or the like and thus requires a complete removal of the substances, that is, the chaotropic reagent or ethanol, so it could be an onerous task and takes a longer time. Recently, International Publication No. WO 00/21973 discloses a method of directly purifying a nucleic acid with a filter. The method involves passing a specimen through the filter to retain cells adsorbed by the filter, dissolving the adsorbed cells, filtering the cells, and then washing and eluting the adsorbed nucleic acid. However, in order to obtain the nucleic acid after absorbing the cells with the filter, the method further requires the selection of the filter depending on the type of the cells. Another disadvantage is that the devices used in this method are too large and complicated for the worker to use with ease.

### DISCLOSURE

### Technical Problem

In view of the aforementioned problems of the conventional nucleic acid extraction techniques, the present disclosure provides a microfluidic chip for extracting nucleic acids, a nucleic acid extraction device having the same, and a nucleic acid extraction method using the same that is able to provide a micro-miniaturization and an ultra-high speed, while maintaining and/or improving reliable nucleic acid extraction efficiencies.

### Technical Solution

According to one aspect of the present disclosure, a microfluidic chip for extracting nucleic acids from a biological sample is provided. The microfluidic chip includes an inlet portion, a heater disposed on a first channel region connected to the inlet portion configured to transmit the heat applied from an outside to the biological sample introduced from the inlet portion, a first filter disposed on a second channel region connected to the heater and configured to filter a substance out wherein the substance has a size larger than a size of the nucleic acids, a nucleic acid separator disposed on a third channel region connected to the first filter and having nucleic acid binding substances capable of specifically binding with the nucleic acids, a second filter disposed on a fourth channel region connected to the nucleic acid separator so as to filter the substance out, and an outlet portion connected to the second filter.

The first channel region, the second channel region, the third channel region, and the fourth channel region are configured to allow a fluid to pass through and have a depth in a range of 0.001 to 10 mm, respectively.

The first filter and the second filter have a thickness in a range of 0.01 to 10 mm, while having pores in a diameter range of 0.1 to 0.4 μm.

The first filter and the second filter have a thickness in a range of 0.01 to 0.5 mm, while having pores in a diameter of 0.2 μm.

The nucleic acid separator has beads to which nucleic acid binding functional groups are attached, as nucleic acid binding substances.

The beads to which the nucleic acid binding functional groups are attached are in a diameter range from 0.001 to 20 mm.

The nucleic acid separator comprises beads to which nucleic acid binding functional groups are attached in a range of 1 μg to 200 mg.

The microfluidic chip is made of a plastic material.

The microfluidic chip further includes a first plate, a second plate disposed on a first side of the first plate and having a channel covering from the first channel region to the fourth channel region, and a third plate disposed on a first side of the second plate and having the inlet portion and the outlet portion.

Each of the first plate and the third plate includes a material selected from the group consisting of polydimethylsiloxane (PDMS), cyclo-olefin copolymer (COC), polymethylmetharcylate (PMMA), polycarbonate (PC), polypropylene carbonate (PPC), polyether sulfone (PES), polyethylene terephthalate (PET), and a combination thereof. The second plate includes a thermoplastic resin or thermosetting resin selected from the group consisting of polymethylmetharcylate (PMMA), polycarbonate (PC), cyclo-olefin copolymer (COC), polyamide (PA), polyethylene (PE), polypropylene (PP), polyphenylene ether (PPE), polystyrene (PS), polyoxymethylene (POM), polyetheretherketone (PEEK), polytetrafluoroethylene (PTFE), polyvinylchloride (PVC), polyvinylidene fluoride (PVDF), polybutyleneterephthalate (PBT), fluorinated ethylenepropylene (FEP), perfluoralkoxyalkane (PFA), and a combination thereof.

The inlet portion on the third plate has a diameter in a range from 0.1 to 5.0 mm. The outlet portion has a diameter in a range from 0.1 to 5.0 mm. Each of the first plate and the third plate has a thickness of 0.01 to 20 mm. The second plate has a thickness of 30 μm to 10 mm.

According to another aspect of the present disclosure, a device for extracting nucleic acids from a biological sample is provided. The device includes a microfluidic chip. The microfluidic chip includes an inlet portion, a heater disposed on a first channel region connected to the inlet portion configured to transmit the heat applied from an outside to the biological sample introduced from the inlet portion, a first filter disposed on a second channel region connected to the heater and configured to filter a substance out wherein the substance has a size larger than a size of the nucleic acids, a nucleic acid separator disposed on a third channel region connected to the first filter and having nucleic acid binding substances capable of specifically binding with the nucleic acids, a second filter disposed on a fourth channel region connected to the nucleic acid separator so as to filter the substance out, and an outlet portion connected to the second filter. The device includes a chip mounting module for mounting the microfluidic chip thereon, a heating module for applying heat to the heater of the microfluidic chip mounted on the chip mounting module, and a fluid control module connected to the inlet portion and/or the outlet portion of the microfluidic chip mounted on the chip mounting module so as to introduce a nucleic acid extraction solution into the microfluidic chip and/or to discharge the solution existing in the microfluidic chip to an outside of the microfluidic chip.

According to the other aspect of the present disclosure, a method for extracting nucleic acids from a biological sample is provided. The method includes providing a microfluidic chip, wherein the microfluidic chip includes an inlet portion, a heater disposed on a first channel region connected to the inlet portion configured to transmit the heat applied from an outside to the biological sample introduced from the inlet portion, a first filter disposed on a second channel region connected to the heater and configured to filter a substance out, wherein the substance has a size larger than a size of the nucleic acids, a nucleic acid separator disposed on a third channel region connected to the first filter and having nucleic acid binding substances capable of specifically binding with the nucleic acids, a second filter disposed on a fourth channel region connected to the nucleic acid separator so as to filter the substance out, and an outlet portion connected to the second filter, introducing the biological sample selected from the group consisting of cells, bacteria and viruses into the inlet portion of the microfluidic chip, moving the introduced biological sample to the heater of the microfluidic chip and performing the lysis of the biological sample by the application of heat to the heater of the microfluidic chip, moving the substances obtained after the biological sample lysis step to the first filter of the microfluidic chip so as to allow the substances to pass through the first filter, and removing the substances not passing through the first filter, moving the substances passing through the first filter to the nucleic acid separator of the microfluidic chip, binding the nucleic acids of the substances passing through the first filter to the nucleic acid binding substances, and removing the substances not binding to the nucleic acid binding substances, separating the nucleic acids from the nucleic acid binding substances, moving the separated nucleic acids to the second filter, and filtering the nucleic acids through the second filter, and moving the substances passing through the second filter to the outlet portion and extracting the nucleic acids from the outlet portion.

### Advantageous Effects

According to one aspect of the present disclosure, the microfluidic chip for extracting nucleic acids, the nucleic acid extraction device having the same, and the nucleic acid extraction method using the same is efficiently able to be associated with PCR, thus being widely applicable in various fields such as diagnosis, treatment and prevention of diseases.

### DESCRIPTION OF DRAWINGS

FIG. 1 is a plan view showing a configuration of a microfluidic chip for extracting nucleic acids according to one embodiment of the present disclosure.

FIG. 2 is a cross section view and a plan view of the microfluidic chip.

FIG. 3 is a block diagram showing a nucleic acid extraction device according to the embodiment of the present disclosure, on which the microfluidic chip for extracting nucleic acids is mounted.

FIG. 4 is a flow chart showing a nucleic acid extraction method according to another embodiment of the present disclosure.

FIGS. 5a and 5b shows comparison results between a general nucleic acid extraction method and a nucleic acid extraction method according to another embodiment of the present disclosure.

FIGS. 6a and 6b shows gel electrophoresis results of the nucleic acids obtained through the method according to another embodiment of the present disclosure and amplified in a first PCR device made in a same manner described in U.S. patent application Ser. No. 13/642,877, and a second PCR device made in a different manner therefrom.

### MODE FOR INVENTION

Before the present invention is disclosed and described, however, it is to be understood that the disclosed embodiments are merely exemplary of the invention, which can be embodied in various forms.

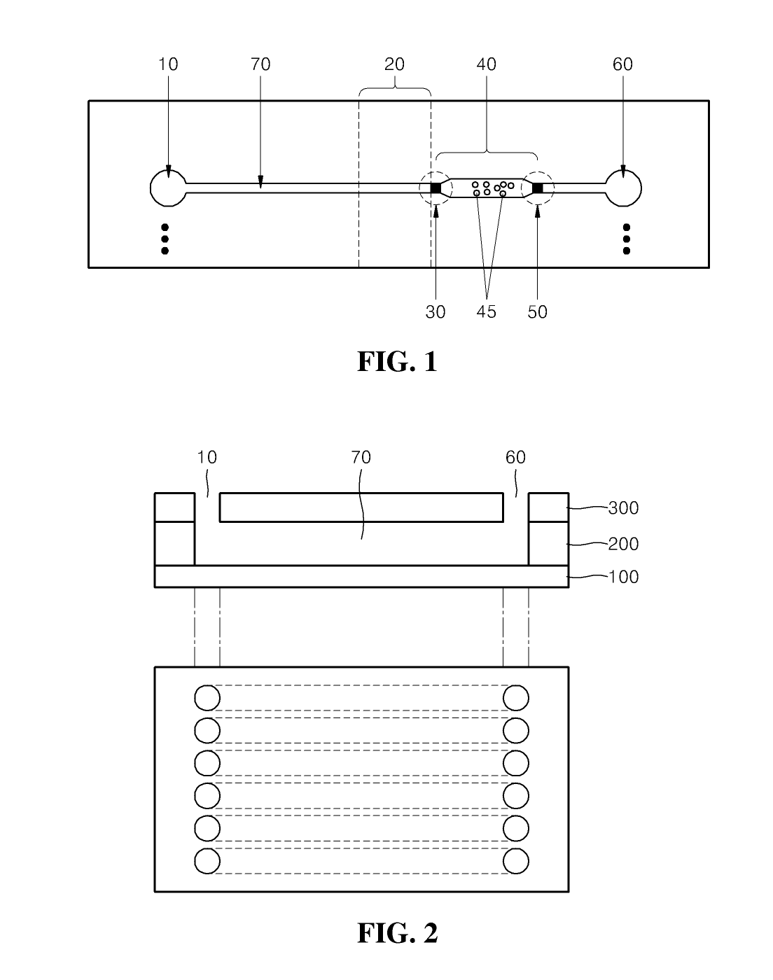

FIG. 1 is a plan view showing a configuration of a microfluidic chip for extracting nucleic acids according to one embodiment of the present disclosure.

Referring to FIG. 1, a microfluidic chip for nucleic acid extraction, according to one embodiment of the present disclosure, extracting a nucleic acid from a biological sample, includes an inlet portion 10, a heater 20 disposed on a first channel region connected to the inlet portion 10 so as to transmit the heat applied from an exterior to the biological sample introduced from the inlet portion 10, a first filter 30 disposed on a second channel region connected to the heater 20 so as to pass substances that have corresponding sizes of the nucleic acids, a nucleic acid separator 40 disposed on a third channel region connected to the first filter 30 and having nucleic acid binding substances 45 binding specifically to the nucleic acids, a second filter 50 disposed on a fourth channel region connected to the nucleic acid separator 40 so as to pass the substances having the corresponding sizes of the nucleic acids, and an outlet portion 60 connected to the second filter 50.

The microfluidic chip for extracting nucleic acids as used herein refers to a microchip that includes the inlet portion, the outlet portion, the channel connecting the inlet portion and the outlet portion with each other, the first filter, and the second filter, which are embodied with a size in a millimeter (mm) or micrometer (μm).

The biological sample as used herein is a biological substance containing a nucleic acid such as Deoxyribonucleic acid (DNA) or Ribonucleic acid (RNA), or the like. For example, the biological sample is a liquid sample containing but not limited to animal cells, plant cells, pathogenic bacteria, fungi, bacteria, viruses and the like.

The inlet portion 10 is a portion at which the biological sample or a nucleic acid extraction solution is introduced into the microfluidic chip, and the outlet portion 60 is a portion at which the nucleic acids obtained from the biological sample, the nucleic acid extraction solution, and other waste are discharged to the exterior of the microfluidic chip. In some cases, the inlet portion 10 may serve as the outlet portion, and the outlet portion 60 as the inlet portion. The solution for nucleic acid extraction includes any solution required for extracting a nucleic acid, such as distilled water, a nucleic acid binding buffer, and an elution buffer. On the other hand, the inlet portion 10 and the outlet portion 60 are connected to each other via a channel 70 so that a fluid can move between them. The components, such as the heater 20, the first filter 30, the nucleic acid separator 40, and the second filter 50, which will be described in detail later, are arranged in the channel 70, and are configured to perform their respective functions. The channel 70 may be embodied in various sizes, and each of the width and depth of the channel 70 is desirably in a range of 0.001 to 10 mm. The first, second, third and fourth channel regions are used to merely refer to a sequential order of regions of the microfluidic chip 1 from the inlet portion 10 up to the outlet portion 60 and are not used to limit to or indicate specific locations in the channel 70.

The heater 20 serves to apply the heat obtained from the exterior to the solution (including the biological sample) introduced into the inlet portion 10 and is disposed on the first channel region connected to the inlet portion 10. For example, if the biological sample containing cells, bacteria, or viruses is introduced through the inlet portion 10, the cells, bacteria, or viruses are heated momentarily up to about 80 to 1000 upon arrival at the heating portion 20, their outer cell membranes are broken and cellular components are released to the outside so that cell lysis can be performed. The heater 20 receives the heat from a heating module 600 of a nucleic acid extraction device as will be described later in a contact or non-contact manner.

The first filter 30 is a structure having given-sized pores and serves to filter out pass-through and non-pass-through substances according to their size of the substances in the direction of the flow of a fluid. In one embodiment of the present disclosure, the first filter 30 is disposed on the second channel region connected to the heater 20 and adapted to allow substances having an equivalent sizes of the nucleic acid to pass through. While substances having the sizes greater than the nucleic acids from the lysates produced via the heating are being collected on the heater 20, the first filter 30 filters the nucleic acids and the substances having the corresponding sizes to the nucleic acids and moves them to the nucleic acid separator 40. The first filter 30 may be embodied in various sizes, and it desirably has a thickness in the range of 0.01 to 10 mm, while having the pores having the diameter in the range of 0.1 to 0.4 μm. More desirably, the first filter 30 has a thickness in a range of 0.01 to 0.5 mm, while having the pores having the diameter of 0.2 μm.

The nucleic acid separator 40 is adapted to selectively separate the nucleic acids from the nucleic acids and the substances having the corresponding sizes to the nucleic acids filtered through the first filter 30. As shown in FIG. 1, the nucleic acid separator 40 is formed in the space between the first filter 30 and the second filter 50 as will be described later, and has the nucleic acid binding substances 45 capable of specifically combined with the nucleic acid. The nucleic acid binding substance 45 includes all and any material that is specifically to the nucleic acids. The nucleic acid binding substances 45 could be binding beads with a nucleic acid binding functional group and may be, for example, a silica (SiO2) beads, and biotin or streptavidin-coated beads. The beads to which the nucleic acid binding functional groups are attached may have various sizes, but desirably, ranging from 0.001 to 20 mm in a diameter. Further, the nucleic acid separator 40 may have various contents of the beads to which the nucleic acid binding functional groups are attached, but desirably, the content of the beads are in the range of 1 μg to 200 mg. After the nucleic acids bind specifically to the nucleic acid binding substances 45, the interior of the nucleic acid separator 40 is cleaned to remove the foreign substances therefrom, and accordingly, the complexes of target nucleic acids and the nucleic acid binding substances 45 remain in the nucleic acid separator 40. After that, if the elution buffer is supplied to the nucleic acid separator 40, the target nucleic acids are separated from the complexes.

The second filter 50 has a structure having given-sized pores, like the first filter 30, and serves to filter out pass-through and non-pass-through substances according to the sizes of the substances in the direction of the flow of the fluid. In one embodiment of the present disclosure, the second filter 50 is disposed on the fourth channel region connected to the nucleic acid separator 40 and adapted to allow the substances having an equivalent size of the nucleic acids to pass through. While the nucleic acid binding substances 45 are being collected in the nucleic acid separator 40, the second filter 50 filters the nucleic acids out from the nucleic acid binding substances 45 and passes them onto the outlet portion 60. The second filter 50 may have various sizes, and desirably has a thickness in a range of 0.01 to 0.5 mm, while having the pores having the diameter in the range of 0.1 to 0.4 μm. More desirably, the second filter 50 has a thickness of 0.3 mm, while having the pores having the diameter of 0.2 μm.

FIG. 2 is a cross section view and a plan view of the microfluidic chip. As shown in FIG. 2, the microfluidic chip for extracting nucleic acids according to one embodiment of the present disclosure includes a first plate 100, a second plate 200 disposed on top of the first plate 100 in such a manner as to form the channel 70 on which the first to fourth channel regions are formed thereon, and a third plate 300 disposed on top of the second plate 200 in such a manner as to form the inlet portion 10 and the outlet portion 60 thereon. According to the embodiment of the present disclosure, the microfluidic chip for extracting nucleic acids may be made of various materials, desirably, a plastic material. If the microfluidic chip is made of a plastic material, heat transmission efficiencies can be improved just with the control of the thickness of the plastic, with the simple production process, thus greatly reducing the manufacturing cost. On the other hand, each of the first plate 100 and the third plate 300 includes a material selected from the group consisting of polydimethylsiloxane (PDMS), cyclo-olefin copolymer (COC), polymethylmetharcylate (PMMA), polycarbonate (PC), polypropylene carbonate (PPC), polyether sulfone (PES), polyethylene terephthalate (PET), and a combination thereof, and the second plate 200 includes a thermoplastic resin or thermosetting resin selected from the group consisting of polymethylmetharcylate (PMMA), polycarbonate (PC), cyclo-olefin copolymer (COC), polyamide (PA), polyethylene (PE), polypropylene (PP), polyphenylene ether (PPE), polystyrene (PS), polyoxymethylene (POM), polyetheretherketone (PEEK), polytetrafluoroethylene (PTFE), polyvinylchloride (PVC), polyvinylidene fluoride (PVDF), polybutyleneterephthalate (PBT), fluorinated ethylenepropylene (FEP), perfluoralkoxyalkane (PFA), and a combination thereof. Further, the inlet portion 10 of the third plate 300 has a diameter in a range of 0.1 to 5.0 mm, the outlet portion 60 having a diameter of 0.1 to 5.0 mm, and each of the first plate 100 and the third plate 300 has a thickness of 0.01 to 20 mm, the second plate 200 having a thickness of 30 μm to 10 mm. If necessary, the microfluidic chip for extracting nucleic acids according to the embodiment of the present disclosure may include at least two inlet portions 10 and outlet portions 20 and the channels connecting the two or more inlet portions and outlet portions, and in this case, the nucleic acids can be extracted from two or more biological samples on a single chip, thus rapidly and efficiently performing the nucleic acid extraction.

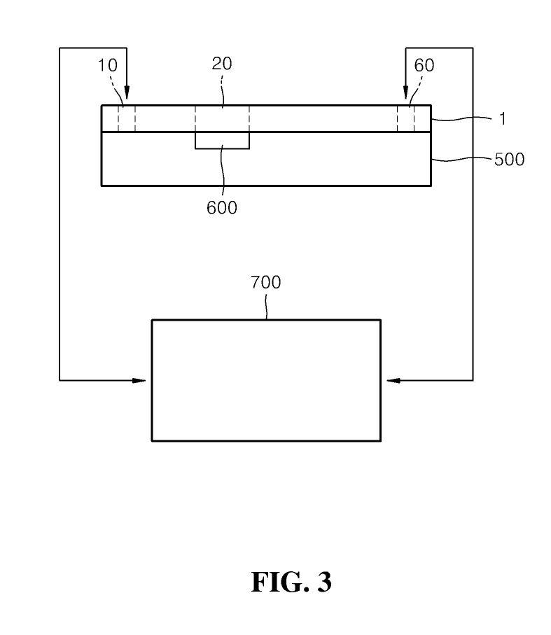

FIG. 3 is a block diagram showing a nucleic acid extraction device according to the embodiment of the present disclosure, on which the microfluidic chip for extracting nucleic acids is mounted.

As shown, the nucleic acid extraction device according to the embodiment of the present disclosure includes the microfluidic chip 1 for extracting nucleic acids, a chip mounting module 500 for mounting the microfluidic chip 1 thereon, a heating module 600 for applying heat to the heater 20 of the microfluidic chip 1 mounted on the chip mounting module 500, and a fluid control module 700 connected to the inlet portion 10 and/or the outlet portion 60 of the microfluidic chip 1 mounted on the chip mounting module 500 so as to introduce the nucleic acid extraction solution into the microfluidic chip 1 and/or to discharge the solution existing in the microfluidic chip 1 to the outside of the microfluidic chip 1.

The nucleic acid extraction device performs all of steps for extracting the nucleic acids in the state where the microfluidic chip 1 is mounted and further includes various modules required for the nucleic acid extraction, in addition to the chip mounting module 500, the heating module 600 and the fluid control module 700. Further, the nucleic acid extraction device according to the embodiment of the present disclosure is configured to conduct all of the steps in an automatic manner and can immediately perform nucleic acid amplification reaction after the extraction of the nucleic acids in association with a PCR device.

The microfluidic chip 1 for extracting nucleic acids has been described herein. The chip mounting module 500 is a part at which the microfluidic chip 1 is mounted. The chip mounting module 500 may have various shapes corresponding to the shapes of the contacted surface of the microfluidic chip 1.

The heating module 600 supplies the heat to the heater 20 of the microfluidic chip 1 when the microfluidic chip 1 is mounted on the chip mounting module 500. The heating module 600 is provided in various forms, desirably, in a form of a contact type heating block.

The fluid control module 700 is connected to the inlet portion 10 and/or the outlet portion 60 of the microfluidic chip 1 mounted on the chip mounting module 500 so as to introduce the nucleic acid extraction solution into the microfluidic chip 1 and/or to discharge the solution existing in the microfluidic chip 1 to the outside of the microfluidic chip 1. The fluid control module 700 may include various components, such as a fine channel as a passage along which the fluid moves, a pneumatic pump providing a driving force with which the fluid moves, a valve controlling the opening and closing of the fluid movement, and a storage chamber for storing various solutions required for the extraction of nucleic acids, such as a nucleic acid binding buffer, an elution buffer, silica gel, and distilled water (DW).

On the other hand, the nucleic acid extraction device according to the embodiment of the present disclosure further includes an electronic control module (not shown) for automatically controlling the microfluidic chip 1, the heating module 600 and the fluid control module 700. The electronic control module can precisely control the modules so as to extract a given amount of nucleic acids from the microfluidic chip 1 according to a previously stored program. The previously stored program includes the program having a series of steps for extracting nucleic acids as will be described below.

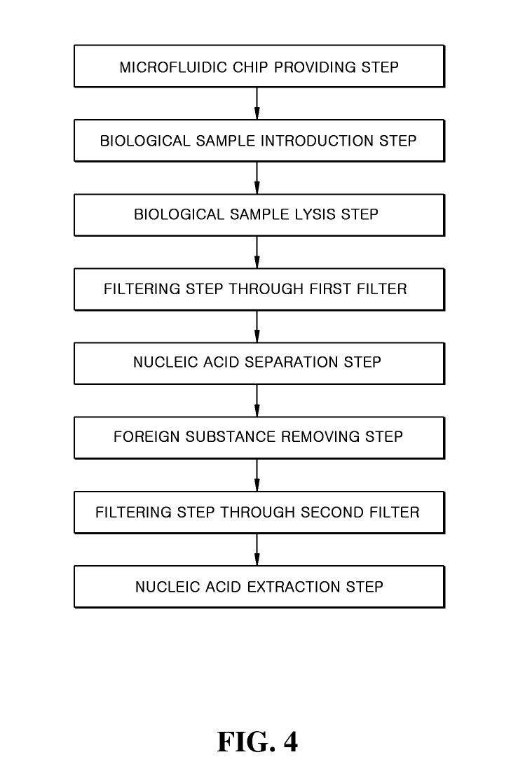

FIG. 4 is a flow chart showing a nucleic acid extraction method according to another embodiment of the present disclosure.

As shown, a nucleic acid extraction method according to another embodiment of the present disclosure uses the microfluidic chip 1 described above.

In more detail, a method for extracting nucleic acids from a biological sample according to another embodiment of the present disclosure includes the steps of: providing the microfluidic chip 1 for extracting the nucleic acids (a microfluidic chip providing step); introducing the biological sample selected from the group consisting of cells, bacteria and viruses into the inlet portion of the microfluidic chip 1 (a biological sample introduction step), moving the introduced biological sample to the heater 20 of the microfluidic chip 1 and performing the lysis of the biological sample by the application of heat to the heater 20 of the microfluidic chip 1 (a biological sample lysis step), moving the substances obtained after the biological sample lysis step to the first filter 30 of the microfluidic chip 1 so as to allow the substances to pass through the first filter 30 and removing the substances not passing through the first filter 30 (a filtering step through the first filter 30), moving the substances passing through the first filter 30 to the nucleic acid separator 40 of the microfluidic chip 1, binding the nucleic acids of the substances passing through the first filter 30 to the nucleic acid binding substances 45, and removing the substances not binding to the nucleic acid binding substances 45 (a nucleic acid separation step), separating the nucleic acids from the nucleic acid binding substances, moving the separated nucleic acids to the second filter 50, and filtering the nucleic acids through the second filter 50 (a filtering step through the second filter 50), and moving the substances passing through the second filter 50 to the outlet portion 60 and extracting the nucleic acids from the outlet portion 60 (a nucleic acid extraction step).

Hereinafter, a quantity of nucleic acid extracted from the biological sample, the time consumed for the extraction, and the reliability in the results of the nucleic acids through PCR (Polymerase Chain Reaction) will be described with reference to first to third embodiments.

### First Embodiment

### Checking the Quantity of Nucleic Acid Extracted from Biological Sample and the Time Consumed for the Extraction

After DNA was first extracted from Mycobacterium tuberculosis cells by using a general tube and by using the microfluidic chip for extracting nucleic acids according to the embodiment of the present disclosure, respectively, the quantity of nucleic acids extracted from the biological sample and the time consumed for the extraction process were observed.

First, the DNA extraction through the general tube are conducted with the following steps.

Mycobacterium tuberculosis cells were prepared, and the prepared Mycobacterium tuberculosis cells, 6% of NaOH, and 4% of NaLC were mixed in the ratio of 1:1:1, thus making a sample solution. After that, the sample solution was subjected to centrifugal separation to remove supernatant liquid (at 4° C. and 4300 rpm for 20 minutes). Next, 1 ml of distilled water was added to the sample solution and subjected to vortexing, and then, the sample solution was moved to another tube. After that, the sample solution was subjected again to centrifugal separation to remove supernatant liquid (at a normal temperature and 12000 rpm for 3 minutes). Next, 1 ml of distilled water was added to the sample solution and subjected to vortexing. After that, 500 μl of distilled water was added to the sample solution to extract genomic DNA (in this case, commercially available QIAamp DNA Kit was used). As a result, about 100 μl of final DNA products were extracted, and the time for the extraction of the final DNA products was more than about one hour.

Now, an explanation on the extraction of nucleic acids from the same Mycobacterium tuberculosis cells through the microfluidic chip for extracting nucleic acids according to the present invention will be given, and the extraction steps are as follows:

Mycobacterium tuberculosis cells were prepared, and the prepared Mycobacterium tuberculosis cells, 6% of NaOH, and 4% of NaLC were mixed in the ratio of 1:1:1, thus making a sample solution. After that, the sample solution was introduced into the inlet portion of the microfluidic chip {25×72×2 mm, silica beads (OPS diagnostics, LLC), and filter (Whatman)} for extracting nucleic acids, as shown in FIG. 1, by means of a syringe (for about one minute). Next, silica gel and 300 μl of 1× DNA binding buffer were introduced into the inlet portion of the microfluidic chip of the present invention, and the heater of the microfluidic chip of the present invention was heated rapidly to 95° C. (for about one minute and 30 seconds). After that, waste was removed from the sample solution through the inlet portion of the microfluidic chip of the present invention, and 100 μl of elution buffer was introduced into the inlet portion of the microfluidic chip of the present invention (for about 30 seconds). Next, final DNA products were extracted through the outlet portion of the microfluidic chip of the present invention. At this time, about 100 μl of final DNA products were extracted, and the time for the extraction of the final DNA products was about seven minutes. If the above-mentioned steps are conducted through automatically operating nucleic acid extraction device, not through a manual operating manner, of course, the time for the extraction is reduced to about 5 minutes and below.

After the first embodiment, it is found that even if the quantity of nucleic acids extracted through the microfluidic chip of the present invention is the same as that extracted through the general tube, the total time consumed for the extraction is very shorter than that in the existing extraction method.

### Second Embodiment

### Results of PCR of DNA Products Extracted Through General Nucleic Acid Extraction Method and Nucleic Acid Extraction Method According to the Present Invention

So as to ensure the reliability of the DNA products extracted through the first embodiment, the extracted DNA products were subjected to PCR. The PCR was conducted by using a PCR device having two heating blocks, as disclosed in Korean Patent Application Laid-open No. 2011-0037352 as filed by the same applicant as the invention, and a commercially available PCR device (Roche, LightCycler). The PCR device as suggested by the same applicant as the invention is a real time PCR device and includes a first heating block disposed on a substrate; a second heating block disposed on the substrate in such a manner as to be spaced apart from the first heating block; and a chip holder movable on the first heating block and the second heating block in left and right and/or up and down by means of driving means and having a PCR chip made of a light transmission plastic material. Further, the driving means includes rails extended in left and right directions and a connection member slidingly movable in the left and right and/or up and down directions along the rails, and the chip holder is disposed on one end of the connection member. Furthermore, a light source is disposed between the first heating block and the second heating block, and a light detector is located on the chip holder so as to detect the light emitted from the light source. Otherwise, a light detector is located between the first heating block and the second heating block so as to detect the light emitted from a light source, and the light source is disposed on the chip holder. In case where the PCR device is used, the PCR time is substantially reduced to about 5 to 15 minutes. If the PCR device is associated with the microfluidic chip for extracting nucleic acids and the nucleic acid extraction device according to the present invention, the nucleic acid extraction time can be substantially reduced to about 5 to 7 minutes, and the time for obtaining the fined amplified nucleic acid products is reduced to at least 20 minutes. On the other hand, in case where the PCR device as suggested by the same applicant as the invention was used to conduct the PCR, total 16 μl of PCR reagent was prepared by mixing 8 μl of real time PCR mixture (NBS SYBR Green I real-time PCR mixture having a concentration of 2×) (final concentration of 1×), 1.6 μl of forward primer having a concentration of 10 μM (final concentration of 1 μM), 1.6 μl of reverse primer having a concentration of 10 μM (final concentration of 1 μM), 3 μl of template DNA, and 1.8 μl (adjusted to 16 μl) of distilled water DW. In case where the PCR device as suggested by another company was used to conduct the PCR, total 20 μl of PCR reagent was prepared by mixing 10 μl of real time PCR mixture (Takara SYBR Green I real-time PCR mixture having a concentration of 2×) (final concentration of 1×), 2 μl of forward primer having a concentration of 10 μM (final concentration of 1 μM), 2 μl of reverse primer having a concentration of 10 μM (final concentration of 1 μM), 3 μl of template DNA, and 3 μl (adjusted to 20 μl) of distilled water DW.

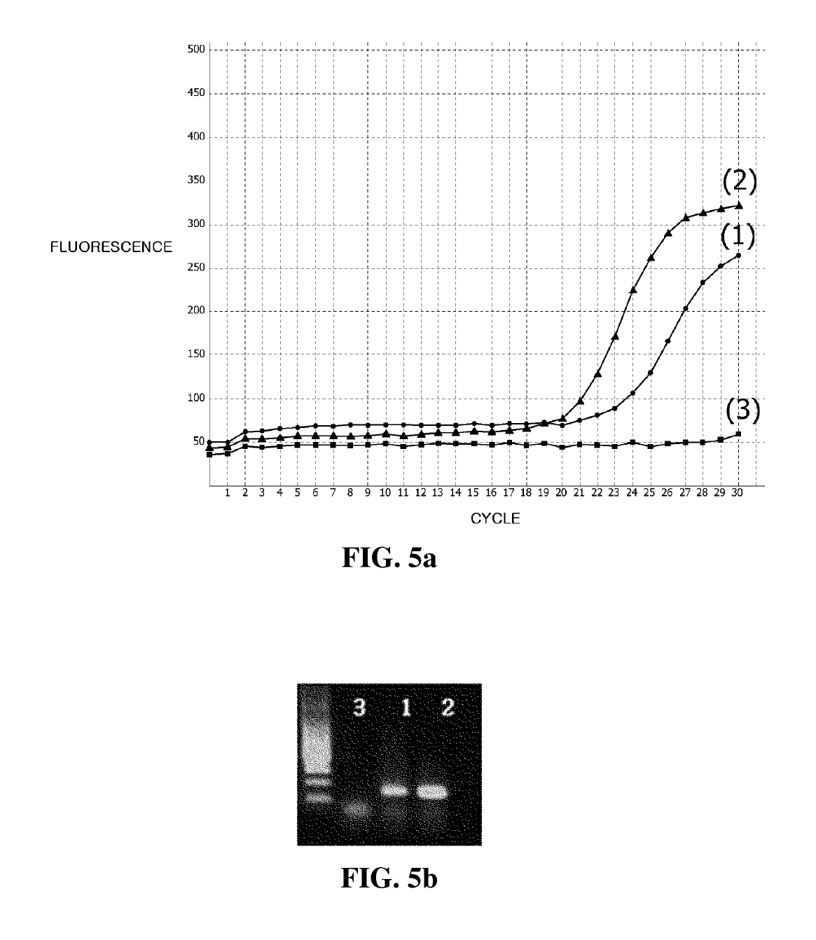

FIGS. 5a and 5b shows comparison results between a general nucleic acid extraction method and a nucleic acid extraction method according to another embodiment of the present disclosure. In more detail, FIG. 5a is a graph showing the degree of fluorescence of the real time PCR results according to PCR cycles using the PCR device as suggested by the same applicant as the invention, and FIG. 5b is a photograph showing gel electrophoresis of the final PCR products.

As shown in FIG. 5a, a curve (1) indicates the PCR result curve (X axis means cycle and Y axis means a degree of fluorescence) of the DNA products through the general nucleic acid extraction method in the first embodiment, a curve (2) indicates the PCR result curve of the DNA products through the nucleic acid extraction method according to the present invention in the first embodiment, and a curve (3) indicates a negative control curve using a solution in which DNA is not contained. As shown, the curves (1) and (2) started to be raised at about cycle 20, unlike the curve (3), so that it was observed that the PCR was appropriately conducted in the first embodiment, and when the curves (1) and (2) were at about cycle 30, they showed the degrees of fluorescence of about 250 and 350, so that it was observed that the DNA products were accurately extracted through the nucleic acid extraction method according to the present invention and further, the quantity of PCR products through the nucleic acid extraction method according to the present invention was more increased than that through the general nucleic acid extraction method. About 15 minutes were needed to complete the extraction of the nucleic acid through the nucleic acid extraction method according to the present invention. Further, as shown in FIG. 5b, a column 1 indicates the PCR result of the DNA products through the general nucleic acid extraction method in the first embodiment, a column 2 indicates the PCR result of the DNA products through the nucleic acid extraction method according to the present invention in the first embodiment, and a column 3 indicates the result of negative control using a solution in which DNA is not contained. The final results as shown in FIG. 5a were observed again through the gel electrophoresis photograph of FIG. 5b. On the other hand, the degrees of fluorescence of the real time PCR results according to PCR cycles were measured by using the PCR device made by another company, and the photograph showing the gel electrophoresis of the final PCR products was observed. The results were almost the same as those in FIGS. 5a and 5b, but about 30 minutes were needed to complete the extraction of the nucleic acid through the PCR device made by another company.

### Third Embodiment

### Results of PCR by Device of DNA Products Extracted Through Nucleic Acid Extraction Method According to the Present Invention

The DNA products extracted through the nucleic acid extraction method according to the present invention were amplified through the PCR device (Roche, LightCycler) made by another company and the PCR device (which is the same as in the second embodiment) as suggested by the same applicant as the invention. In this case, PCR was conducted by the PCR device (Roche, LightCycler) made by another company wherein a pre-denaturation step (95° C.) was carried out with one cycle for two minutes, a denaturation step (95° C.) was carried out with 30 cycle for 10 seconds, and annealing and elongation steps (72° C.) were carried out with 30 cycle for 10 seconds, and on the other hand, PCR was conducted by the PCR device as suggested by the same applicant as the invention wherein a pre-denaturation step (95° C.) was carried out with one cycle for 8 seconds, a denaturation step (95° C.) was carried out with 30 cycle for 8 seconds, and annealing and elongation steps (72° C.) were carried out with 30 cycle for 14 seconds.

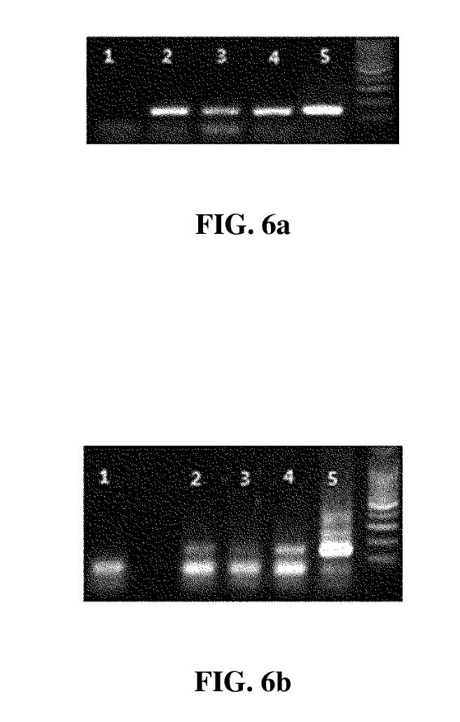

FIGS. 6a and 6b shows gel electrophoresis results of the nucleic acids obtained through the method according to another embodiment of the present disclosure and amplified in a first PCR device made in a same manner described in U.S. patent application Ser. No. 13/642,877, and a second PCR device made in a different manner therefrom.

In more detail, FIG. 6a is a graph showing the gel electrophoresis results of the final PCR products through the PCR device made by another company, and FIG. 6b is a graph showing the gel electrophoresis results of the final PCR products through the PCR device as suggested by the same applicant as described in U.S. patent application Ser. No. 13/642,877. Further, as shown in FIGS. 6a and 6b, a column 1 indicates the result of negative control using a solution in which DNA is not contained, columns 2 to 4 indicate the final PCR products using the DNA products extracted from 200 μl of Mycobacterium tuberculosis cells through the nucleic acid extraction method according to the present invention, and a column 5 indicates the final PCR products from 1 ml of Mycobacterium tuberculosis cells through the commercially available QIAamp DNA Kit. As shown in FIGS. 6a and 6b, it was observed that even if the PCR is conducted by using the different PCR devices, the PCR results are almost similar to each other. However, the time consumed for the PCR through the PCR device as suggested by the same applicant as the invention is greatly reduced (wherein the time consumed for the completion of the PCR through the PCR device made by another company is about 30 minutes, and the time consumed for the completion of the PCR through the PCR device as suggested by the same applicant as the invention is about 15 minutes), while reliable nucleic acid extraction results are being provided.

While the present invention has been described with reference to the particular illustrative embodiments, it is not to be restricted by the embodiment but only by the appended claims. It is to be appreciated that those skilled in the art can change or modify the embodiments without departing from the scope and spirit of the present invention.

1-13. (canceled)

14. A microfluidic chip for extracting nucleic acids from a biological sample, the microfluidic chip comprising:
an inlet portion;
a heater disposed on a first channel region connected to the inlet portion configured to transmit the heat applied from an outside to the biological sample introduced from the inlet portion;
a first filter disposed on a second channel region connected to the heater and configured to filter a substance out wherein the substance has a size larger than a size of the nucleic acids;
a nucleic acid separator disposed on a third channel region connected to the first filter and having nucleic acid binding substances capable of specifically binding with the nucleic acids;
a second filter disposed on a fourth channel region connected to the nucleic acid separator so as to filter the substance out; and
an outlet portion connected to the second filter.

15. The microfluidic chip according to claim 14, wherein the first channel region, the second channel region, the third channel region, and the fourth channel region are configured to allow a fluid to pass through and have a depth in a range of 0.001 to 10 mm, respectively.

16. The microfluidic chip according to claim 14, wherein the first filter and the second filter have a thickness in a range of 0.01 to 10 mm, while having pores in a diameter range of 0.1 to 0.4 μm.

17. The microfluidic chip according to claim 14, wherein the first filter and the second filter have a thickness in a range of 0.01 to 0.5 mm, while having pores in a diameter of 0.2 μm.

18. The microfluidic chip according to claim 14, wherein the nucleic acid separator has beads to which nucleic acid binding functional groups are attached, as nucleic acid binding substances.

19. The microfluidic chip according to claim 18, wherein the beads to which the nucleic acid binding functional groups are attached are in a diameter range from 0.001 to 20 mm.

20. The microfluidic chip according to claim 18, wherein the nucleic acid separator comprises beads to which nucleic acid binding functional groups are attached in a range of 1 μg to 200 mg.

21. The microfluidic chip according to claim 14, wherein the microfluidic chip is made of a plastic material.

22. The microfluidic chip according to claim 14, wherein the microfluidic chip further comprises,
a first plate;
a second plate disposed on a first side of the first plate and having a channel covering from the first channel region to the fourth channel region; and
a third plate disposed on a first side of the second plate and having the inlet portion and the outlet portion.

23. The microfluidic chip according to claim 22, wherein each of the first plate and the third plate comprises a material selected from the group consisting of polydimethylsiloxane (PDMS), cyclo-olefin copolymer (COC), polymethylmetharcylate (PMMA), polycarbonate (PC), polypropylene carbonate (PPC), polyether sulfone (PES), polyethylene terephthalate (PET), and a combination thereof, and
wherein the second plate comprises a thermoplastic resin or thermosetting resin selected from the group consisting of polymethylmetharcylate (PMMA), polycarbonate (PC), cyclo-olefin copolymer (COC), polyamide (PA), polyethylene (PE), polypropylene (PP), polyphenylene ether (PPE), polystyrene (PS), polyoxymethylene (POM), polyetheretherketone (PEEK), polytetrafluoroethylene (PTFE), polyvinylchloride (PVC), polyvinylidene fluoride (PVDF), polybutyleneterephthalate (PBT), fluorinated ethylenepropylene (FEP), perfluoralkoxyalkane (PFA), and a combination thereof.

24. The microfluidic chip according to claim 22, wherein the inlet portion on the third plate has a diameter in a range from 0.1 to 5.0 mm,
wherein the outlet portion has a diameter in a range from 0.1 to 5.0 mm,
wherein each of the first plate and the third plate has a thickness of 0.01 to 20 mm, and
wherein the second plate has a thickness of 30 μm to 10 mm.

25. A device for extracting nucleic acids from a biological sample, the device comprising:
a microfluidic chip,
wherein the microfluidic chip comprising an inlet portion, a heater disposed on a first channel region connected to the inlet portion configured to transmit the heat applied from an outside to the biological sample introduced from the inlet portion, a first filter disposed on a second channel region connected to the heater and configured to filter a substance out wherein the substance has a size larger than a size of the nucleic acids, a nucleic acid separator disposed on a third channel region connected to the first filter and having nucleic acid binding substances capable of specifically binding with the nucleic acids, a second filter disposed on a fourth channel region connected to the nucleic acid separator so as to filter the substance out, and an outlet portion connected to the second filter;
a chip mounting module for mounting the microfluidic chip thereon;
a heating module for applying heat to the heater of the microfluidic chip mounted on the chip mounting module; and
a fluid control module connected to the inlet portion and/or the outlet portion of the microfluidic chip mounted on the chip mounting module so as to introduce a nucleic acid extraction solution into the microfluidic chip and/or to discharge the solution existing in the microfluidic chip to an outside of the microfluidic chip.

26. A method for extracting nucleic acids from a biological sample, the method comprising:
providing a microfluidic chip,
wherein the microfluidic chip comprising an inlet portion, a heater disposed on a first channel region connected to the inlet portion configured to transmit the heat applied from an outside to the biological sample introduced from the inlet portion, a first filter disposed on a second channel region connected to the heater and configured to filter a substance out wherein the substance has a size larger than a size of the nucleic acids, a nucleic acid separator disposed on a third channel region connected to the first filter and having nucleic acid binding substances capable of specifically binding with the nucleic acids, a second filter disposed on a fourth channel region connected to the nucleic acid separator so as to filter the substance out, and an outlet portion connected to the second filter;
introducing the biological sample selected from the group consisting of cells, bacteria and viruses into the inlet portion of the microfluidic chip;
moving the introduced biological sample to the heater of the microfluidic chip and performing the lysis of the biological sample by the application of heat to the heater of the microfluidic chip;
moving the substances obtained after the biological sample lysis step to the first filter of the microfluidic chip so as to allow the substances to pass through the first filter, and removing the substances not passing through the first filter;
moving the substances passing through the first filter to the nucleic acid separator of the microfluidic chip, binding the nucleic acids of the substances passing through the first filter to the nucleic acid binding substances, and removing the substances not binding to the nucleic acid binding substances;
separating the nucleic acids from the nucleic acid binding substances, moving the separated nucleic acids to the second filter, and filtering the nucleic acids through the second filter; and
moving the substances passing through the second filter to the outlet portion and extracting the nucleic acids from the outlet portion.
